(19) United States
(12) Patent Application Publication
(10) Pub. No.: US 2025/0003900 A1
(43) Pub. Date: Jan. 2, 2025

(54) Micro Magnetic Resonance Relaxometry (MMRR) for Rapid and Non-Invasive Detection of Senescence in Mesenchymal Stem Cells

(71) Applicant: Massachusetts Institute of Technology, Cambridge, MA (US)

(72) Inventors: Jongyoon Han, Bedford, MA (US); Laurie Boyer, Westborough, MA (US); Smitha Surendran Thamarath, Singapore (SG); Shu Hui Neo, Singapore (SG); Ching Ann Tee, Singapore (SG)

(73) Assignee: Massachusetts Institute of Technology, Cambridge, MA (US)

(21) Appl. No.: 18/703,913

(22) PCT Filed: Oct. 27, 2022
(86) PCT No.: PCT/US2022/047982

§ 371 Date: Apr. 23, 2024

Related U.S. Application Data
(60) Provisional application No. 63/272,738, filed on Oct. 28, 2021.

(51) Int. Cl. G01N 24/08; C12N 5/00; G01R 33/50

(52) U.S. Cl. CPC G01N 24/08; C12N 5/0081; G01R 33/50

(57) ABSTRACT

Senescent cells can be detected using magnetic resonance relaxometry.

### CLAIM OF PRIORITY

The application claims priority to U.S. Provisional Patent Application No. 63/272,738, filed Oct. 28, 2021, which is incorporated by reference in its entirety.

### FIELD OF THE INVENTION

The invention features systems and methods for non-invasive detection.

### BACKGROUND

Cellular senescence is a cell state that leads to cell cycle exit that are accompanied by genetic, metabolic, and morphological changes of cells, due to aging and other external or internal conditions. (Ref. 1) Although senescence can be important for preventing cancer initiation and tumor progression (Refs. 2-5), the accumulation of senescent cells in the body can also have harmful effects, especially in age-related diseases like neurodegeneration, cardiovascular disease, osteoarthritis, renal dysfunction, non-alcoholic fatty liver disease, Type 2 diabetes, among others. (Refs. 3, 6 and 7). Cells with senescent characteristics are often present in tissues affected by age-related diseases, and their detection and elimination have become a major target for medical interventions (Ref. 2). In a senescent cell, the expression of genes involved in DNA replication, DNA repair, and cell cycle are downregulated (Ref. 8). In addition, senescent cells display a senescence-associated secretory phenotype (SASP), which causes an alteration in the tissue microenvironment, local or systemic inflammation, disruption in normal tissue structure, leading to resistance to immune clearance of senescent cells (Refs. 3, 6 and 9). Apart from aging, tumor-associated stresses like DNA lesions (Ref. 10), reactive oxygen species (ROS) (Ref. 11), certain oncogene activation (Ref. 12), and exposure of interferon-β (Ref. 13), also induce cellular senescence (Ref. 2).

### SUMMARY

The presence of senescent cells in in vitro culture of adult mesenchymal stromal cells (MSCs) has challenged their clinical utility for regenerative medicine and cell therapy. A non-invasive and rapid method to detect senescent cells in heterogeneous cell cultures using microscale Magnetic Resonance Relaxometry (uMRR) and a system for detection is reported. For example, it is shown that the T₂ relaxation time measured by uMRR is strongly correlated with the proportion of senescent cells under diverse conditions, including different passages and donors, among size-sorted MSCs by inertial spiral microfluidic device, and in MSCs with induced senescence. The μMRR measurements also correlated well with conventional assays for senescence detection that often lead to the destruction of cells. Since the presence of senescent cells can impact the multi-lineage differentiation potential and quality of MSCs, the uMRR assay and system described herein represents a promising, non-invasive, and rapid critical quality analytic for improving the efficacy of MSCs for patients.

In general, a rapid and non-invasive detection of cellular senescence based on the transverse relaxation of protons by using micro magnetic resonance relaxometry (uMRR) is described.

In one aspect, a method of detecting senescent cells can include loading a liquid sample including a plurality of cells in a sensor, placing the sensor including the liquid sample within a detection coil of a magnetic resonance relaxometry device, and determining a T₂ value to detect an amount of senescent cells in the liquid sample. The sensor can be a tube or a chamber.

In another aspect, a method of improving efficacy of stem cells or progenitor cells for patients can include detecting senescent cells in a sample according to the method described herein, and concentrating cells in the sample to reduce a number of the senescent cells in the sample.

In another aspect, a system for detecting senescent cells can include a magnetic resonance relaxometry device configured to detect an amount of senescent cells in a liquid sample, and a cell separation device for reducing the amount of senescent cells in the liquid sample based on output from the magnetic resonance relaxometry device. In certain circumstances, the liquid sample can be taken from a cell batch. The cell separation device can remove senescent cells from the cell batch based on detecting the amount of senescent cells.

In certain circumstances, the magnetic resonance relaxometry device can be configured to determine a T₂ value to detect an amount of senescent cells in the liquid sample.

In certain circumstances, the amount of senescent cells in the liquid sample can be proportional to a ferritin concentration in the liquid sample.

In certain circumstances, the magnetic resonance relaxometry device can be configured to determine a T₂ value to detect an amount of senescent cells includes quantifying an amount of Fe³⁺ in the liquid sample.

In certain circumstances, the amount of senescent cells in the liquid sample can be determined relative to a reference sample.

In certain circumstances, the amount of senescent cells in the liquid sample can be proportional to a ferritin concentration in the liquid sample.

In certain circumstances, determining a T₂ value to detect an amount of senescent cells can include quantifying an amount of Fe³⁺ in the liquid sample.

In certain circumstances, the magnetic resonance relaxometry device can include a radio frequency probe.

In certain circumstances, the T₁ relaxation time can be used in conjunction with T₂ value to determine whether senescent cells are present in the sample.

In certain circumstances, determining the T₁ or T₂ value can include supplying a train of pulses over a period of less than one minute.

In certain circumstances, determining the T₁ or T₂ value can include obtaining and averaging 2 to 70 scans.

In certain circumstances, the T₂ value can decrease as a number of senescent cells increases.

In certain circumstances, the cells can include stem cells or progenitor cells. For example, the stem cells or progenitor cells can include mesenchymal stromal cells (MSCs), hematopoietic stem cells (HSCs), or induced pluripotent stem cells (iPSCs).

In certain circumstances, a detection region of the magnetic resonance relaxometry device can include a volume of less than about 1 μL of the sample. For example, the volume can be less than 0.1 μL, less than about 0.01 μL, less than about 0.001 μL, or less than about 0.0001 μL, In certain circumstances, the volume can be about 1 μL to 10 μL.

In certain circumstances, determining a T₂ can include measuring a content of paramagnetic or ferromagnetic ions of the cells. The paramagnetic or ferromagnetic ions can include iron or copper.

In certain circumstances, concentrating cells can include passing the sample through a cell separation device, thereby separating senescent cells from non-senescent cells in the sample.

In certain circumstances, the cell separation device can include a microfluidic cell sorter, an inertial focusing device, a microfluidic filtration device, a centrifugal flow device, a deterministic lateral displacement (DLD) chip, a tangential flow microfiltration device, or combinations thereof.

In certain circumstances, the cell separation device can include a microfluidic spiral path.

In certain circumstances, the liquid sample can be contained in a microcapillary.

Other aspects, embodiments, and features will be apparent from the following description, the drawings, and the claims.

### BRIEF DESCRIPTION OF DRAWINGS

FIG. 1A depicts a schematic of a Magnetic Resonance Relaxometry (MRR) device.

FIG. 1B depicts a schematic of system including a Magnetic Resonance Relaxometry (MRR) device and a cell separation device.

FIG. 2 depicts a protocol to separate senescent MSCs from culture expansion and its MRR detection. The spiral microfluidic device as shown in the figure has one inlet and two outlets. The MSC culture is pumped into the microfluidic device and sorted in a speed of 3.5 ml/min to collect the Large cells (22-26 μm) from inner outlet and the cells from the outer outlet sorted again at a speed of 1.5 ml/min to collect small and proliferating cells at (11-15 μm) and medium sized cells (15-22 μm). Normalized concentration of all sorted cells are filled in micro capillary tube and analyzed by MRR.

FIG. 3A depicts a schematic showing different behavior of iron ions in normal and senescent cells.

FIG. 3B depicts a pulse sequence that can be used in the systems and methods described herein.

FIG. 3C depicts an MRR system and senescence measurements.

FIG. 4A depicts separation of young/proliferating and senescent MSCs using a spiral microfluidic device from MSC culture expansion. The MSC culture expansion is pumped into the microfluidic device and sorted in various speeds to collect different sized MSCs at the outlets.

FIG. 4B depicts cell diameter (μm) of sorted MSCs using spiral microfluidic sorting device.

FIG. 4C depicts average T₂ values (n=6) of unsorted and size sorted MSCs from two donors using spiral microfluidic device. Large cells (22-26 μm) are the size sorted MSCs collected in first round of sorting and Medium (15-22 μm) & Small (11-15 μm) cells are collected from the 2^{nd} round of sorting. Statistical analysis of unsorted & small cells (n=6, *P=0.001) and unsorted & Large cells (n=6, *P=0.002) are done by 2-tailed T-test.

FIG. 4D depicts mRNA expression level of unsorted and size sorted MSCs showing the overexpression of senescent markers P16 and P21 in Large MSCs.

FIG. 4E depicts Luminex assay of unsorted and size sorted MSCs from two different donors shows overexpression of senescence associated secretory phenotypes.

FIG. 4F depicts β-galactosidase staining of unsorted and size sorted MSCs where the cytoplasm of senescent MSCs are stained as blue (black arrows).

FIG. 5A depicts T₂ values (n=3) of MSCs from different donors D1 to D5 (passage 3).

FIG. 5B depicts multilineage differentiation (Adipogenesis, Osteogenesis and Chondrogenesis) images of respective MSC donors in passage 3. Osteogenesis induction was confirmed with Alizarin Red S staining for calcium deposits, adipogenesis differentiation was determined with Oil Red O staining for the detection of lipid droplets. Chondrogenesis differentiation was identified by glycosaminoglycan staining with Safranin O.

FIG. 5C depicts average T₂ values (n=3) of MSCs which is passaged serially from 3 to 8.

FIG. 5D depicts relative mRNA expression values of P21 and P16 for MSCs from same donor for passage P7 compared to the passage P4 as a control.

FIG. 5E depicts β-galactosidase staining of MSCs from same donor for passage P4, P6 and P8 where cytoplasm of senescent MSCs were stained as blue (black arrows).

FIG. 6A depicts immunofluorescent images of MSCs which is culture expanded as passages P3, P5, P6 and doxorubicin treated MSCs from P3. The cells are stained by the senescence marker (γ-H2aX foci) which emits red fluorescence and the proliferation marker, 5-ethynyl-2′-deoxyuridine (Edu) which emits green fluorescence. The cell nuclei are counterstained with NucBlue (Hoechst 33342) which emits blue fluorescence (shown as DAPI) when bound to DNA.

FIG. 6B depicts β-galactosidase staining of MSCs for passages P3, P5 and P6 where the cytoplasm of senescent MSCs were stained as blue.

FIGS. 6C and 6D depict the proliferation (Pf) Index (%) and Senescent (Sn) Index (%) are calculated by counting number the green and red fluorescent stains relative to the number of blue fluorescent stains in all images. The total of 10 images from each of 3 repetitions of staining experiments in MSCs of P3, P5, P6 and DOX treated were used for the calculation of Pf Index (%) and Sn Index (%) which are plotted in (FIG. 6C) and (FIG. 6D) respectively.

FIG. 6E depicts relative mRNA expression values of senescence-associated markers p16 and p21 for MSCs at different passages of P3, P5 and P6.

FIG. 6F depicts average T₂ results (n=3) of P3, P5, P6 and DOX treated. Statistical analysis of P3 and DOX (n=3) are done by 2-tailed T-test in (FIG. 6C), (FIG. 6D) and (FIG. 6F) and P values are shown in respective graphs.

FIG. 6G depicts average T₂ values (n=3) of MSCs (same donor, Passage 4) control and treated with TGB-β1 (blue) and IL-1 (red) at different concentrations (10 and 20 mg/ml).

FIG. 6H depicts limit of detection of MRR assay for senescent MSCs. Average T₂ values (n=3) of MSCs with increasing cell numbers in micro capillary tube of MRR for control and TGF-β1 (10 and 20 mg/ml) treated MSCs of same donor at passage 4. The T₂ value (n=3) of PBS in microcapillary tube is shown as black bar at the bottom of figure (FIG. 6B).

FIG. 7A depicts T₂ values of unsorted and sorted MSCs. Large cells (22-26 μm) are the sorted MSCs collected in first round of sorting and Medium (15-22 μm) & Small (11-15 μm) cells are collected from the 2nd round of sorting.

FIG. 7B depicts multilineage differentiation (Osteogenesis and Chondrogenesis) images of unsorted and sorted cells.

FIG. 8 depicts limit of detection of MRR for senescent MSCs. T₂ values of different cell concentrations of control and TGF-β1 (10 and 20 mg/ml) treated MSCs (Donor 3, passage 4). The T₂ value of PBS is shown as black bar at the bottom of figure.

### DETAILED DESCRIPTION

Cellular senescence is a cell state that leads to cell cycle exit that are accompanied by genetic, metabolic, and morphological changes of cells, due to aging and other external or internal conditions. (Ref. 1) Apart from aging, tumor-associated stresses like DNA lesions (Ref. 10), reactive oxygen species (ROS) (Ref. 11), certain oncogene activation (Ref. 12), and exposure of interferon-β (Ref. 13), also induce cellular senescence (Ref. 2).

In addition to in vivo senescence observed in tissues, cellular senescence is also observed in cultured cells (Ref. 14). For example, mesenchymal stem/stromal cells (MSCs) have been investigated intensively in many clinical trials for indications in ischemic, inflammatory, autoimmune, regenerative potential, and degenerative disorders (Ref. 9), yet senescent MSCs impose significant challenges in their clinical applications (Refs. 14 and 15). Despite the clinical potential of MSCs, proliferation arrest is observed in MSC populations, along with morphological and phenotype alterations, after prolonged culture in vitro (Refs. 14 and 16-18). Moreover, MSCs isolated from older donors show a higher level of senescence than younger donors (Ref. 19). In senescent MSCs, the cells are enlarged and flat with stiffened nuclei and granular cytoplasm (Refs. 3, 14, 18 and 20). Senescent MSCs have decreased multi-lineage differentiation potential (Refs. 14, 17, 18 and 21) and altered secretory and immunomodulatory functions, resulting in reduced therapeutic value (Refs. 3, 22 and 23). Therefore, it is crucial to determine the senescent state of MSCs and other cell-based products as part of quality control in therapeutic cell production.

The standard method for detecting senescent MSCs is the histochemical staining of cellular senescence biomarker acidic lysosomal β-galactosidase (β-gal) (Refs. 3 and 24). The staining procedure requires stable pH (˜6), cell fixation, and a long incubation period (>12 hours). Alternatively, a modified colony-forming unit assay (CFU-f) can detect cell senescence by estimating the remaining proliferative potential of the MSCs after multiple passages (Refs. 18, 25 and 26). Real-time polymerase chain reaction (qPCR) can also detect senescence by providing the gene expression level of senescence-associated markers p16, p21, p53 (Refs. 3, 6 and 27). However, these assays require lengthy and laborious procedures (Ref. 28). In addition, these methods are destructive end-point assays, which are not adequate for quality control of MSCs during cell manufacturing. Therefore, a rapid detection method that allows real-time quantification of the MSC senescence state is critical for the quality control of cell therapeutics for regenerative medicine.

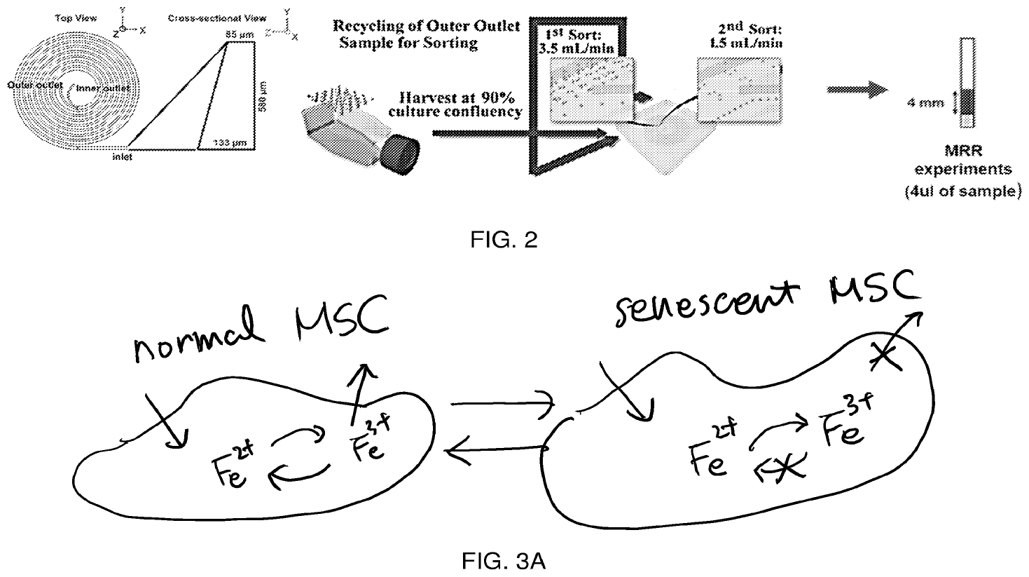

Significant evidence associates iron homeostasis and its dysfunction with aging and related pathologies (Refs. 29-33). Cellular iron is present in Fe²⁺ (reactive, labile, or ‘free’ iron, diamagnetic) or Fe³⁺ (paramagnetic, often bound with iron storage protein ferritin) forms (FIG. 2A). In iron-homeostatic cells, uptake, balancing, and recycling of Fe²⁺/Fe³⁺ are maintained by various iron transport system proteins (e.g., transferrin (Tf), TfR1, ferroportin, NCOA4, DMT1) and regulators (e.g., p53, NRF2) (Ref. 34), involving many different molecular pathways (Ref. 35). Notably, growing evidence indicates that an increase in intracellular iron is a universal hallmark of senescent phenotypes and may serve as a ‘natural marker’ for cellular senescence. Senescent cells accumulate iron (ferritin-bound Fe³⁺) up to ˜30 fold by adjusting their iron homeostasis proteins (Ref. 36) and also by inhibiting ferritinophagy (Ref. 37) (autophagy of Fe³⁺-bound ferritin) that recycles stored iron back to Fe²⁺. This sequestration of iron in ferritin (Fe³⁺) also prevents ferroptosis (Ref. 38), a unique cell death pathway requiring labile iron (Fe²⁺) released from ferritinophagy (Ref. 39). Therefore, directly quantifying intracellular iron (Fe³⁺) could provide a non-invasive measure for the detection of cellular senescence.

Microscale-Magnetic Resonance Relaxometry (uMRR) was previously reported as an efficient malaria diagnostic (Ref. 40). The increased magnetic susceptibility of paramagnetic Fe³⁺ in the hemozoin crystals of infected red blood cells (RBCs) caused faster transverse relaxation of protons (T₂) than the diamagnetic Fe²⁺ state uninfected/healthy RBCs (Refs. 40 and 41). In addition, uMRR-based phenotyping of the oxidative stress response in diabetes mellitus patients' blood was reported as a potential alternative to the conventional A1c test (Ref. 42). Others also reported the high-sensitivity detection of tumor cells (Refs. 43-45), bacteria (Ref. 46), and tuberculosis (Ref. 47) by using a similar uMRR device with immunomagnetic labeling of molecular and cellular targets. As shown herein, uMRR is a non-invasive, non-labeling, and rapid method for detecting senescence in MSCs and could identify MSCs of limited proliferative capacity during MSC manufacturing for cell-based products, opening the door for broader adaptation of this method for detecting cellular senescence in other cells and tissues.

The system and method described herein provides the ability to quantify senescent cells in vitro by a rapid, non-invasive approach that requires a small number of cells (<10⁵), and does not require any reagents or sample preparation steps. Direct Fe³⁺ quantification by a magnetic resonance relaxometry device unexpected provides well-correlated results with conventional end-point and time-intensive assays typically used to measure senescent cells such as qPCR, Luminex assay, and β-galactosidase staining. Measurement of proton resonance relaxation reveals the proportion of senescent cells in a sample. Since paramagnetic Fe³⁺ (but not diamagnetic Fe²⁺) increases the magnetic susceptibility, which stimulates proton nuclear relaxation of water molecules in the cells, this measurement is specifically quantifying the Fe³⁺ content of cellular iron. Previously, intracellular Fe²⁺ (labile iron) was measured by colorimetric and other assays (Ref. 54), yet reliable and quantitative detection of Fe²⁺ has generally been challenging (Refs. 55 and 56), presumably due to the reactive nature of Fe²⁺. The MRR measurements can be used to quantify an amount of Fe³⁺ in a sample. The amount of Fe³⁺ in the sample can be correlated with cellular senescence.

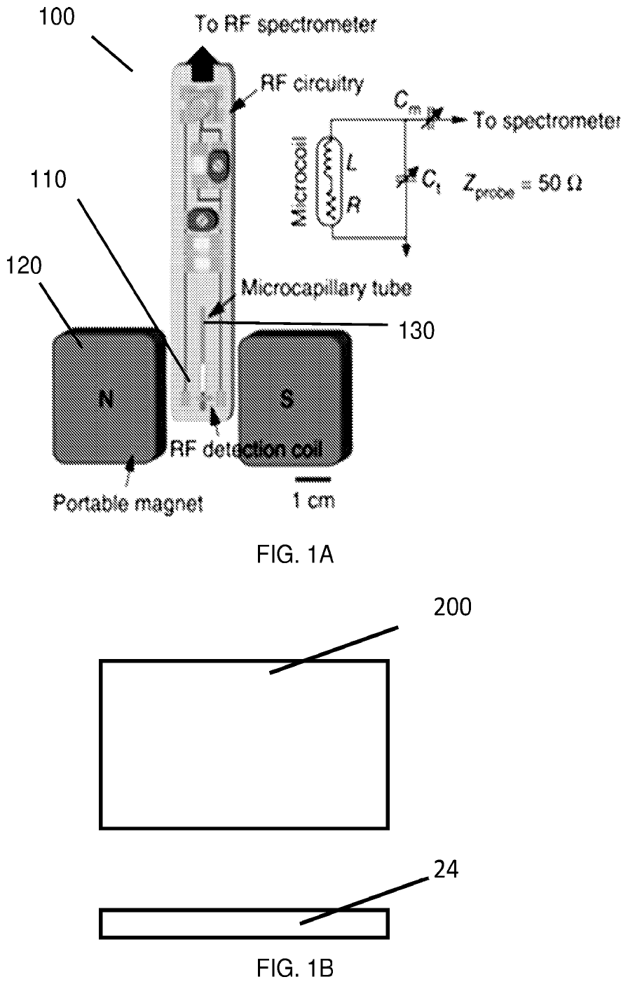

A device for performing magnetic resonance relaxometry is described, for example, in U.S. Pat. No. 10,429,467, which is incorporated by reference in its entirety. Referring to FIG. 1A, a device can include an MRR system. FIG. 1A is a schematic of a Magnetic Resonance Relaxometry (MRR) system 100 in accordance with one aspect of this disclosure. The system 100 can include a Field-Programmable Gate Array-based (FPGA-based) radio frequency (rf) spectrometer to control the MRR system 100, a first direct digital synthesis module for generation of radio frequency pulses, a transmitter (TRANS) for transmission of the generated radio frequency pulses to a radio frequency (rf) probe and detection coil 110, a receiver (RCVR) for receiving resonance information from the radio frequency probe, a first power amplifier (PA), a pre-amplifier (p-amp), a duplexer (Dup) for transmitting a high power excitation pulse to the rf probe in the transmission mode and for isolating the high power excitation pulse from the receiver during receiving mode, and a magnet system 120. A sample 130 can be placed in a sensor, such as a tube or chamber, for example, a microcapillary tube, that can be positioned in an RF detection coil. In many embodiments, the FPGA-based rf spectrometer can include a pulse programmer (PPG) adapted to control the FPGA-based rf spectrometer and a second direct digital synthesis (DDS). The second DDS can generate a fixed intermediate frequency (IF). The first DDS can be configured to generate a variable desired frequency. In accordance with one aspect of this disclosure, the FPGA-based spectrometer may use the design set forth in Takeda K. (2007), “A highly integrated FPGA-based nuclear magnetic resonance spectrometer,” Rev Sci Instrum 78 (3): 033103; and/or in Takeda K. (2008) “OPENCORE NMR: open-source core modules for implementing an integrated FPGA-based NMR spectrometer,” Journal of Magnetic Resonance 192 (2): 218-229, the teachings of which two references are incorporated by reference in their entirety.

In order to facilitate processing of information to and from the MRR system 100, the FPGA-based rf spectrometer is couplable to at least one external electronic device which may, for example, include a personal computer, mobile phone and/or a portable electronic tablet. Coupling between the MRR system 100 and the at least one external electronic device may be by way of at least one of USB, HDMI and/or wireless connection means such as Wi-Fi and/or Bluetooth.

In conventional NMR systems, the major cost of instrumentation lies on the superconducting magnet (or permanent magnet) and rf-spectrometer. In accordance with one aspect of this disclosure, the whole system may cost less than $2500; in which the majority of the cost lies on the FPGA chip ($1000 each), external GHz-clock ($250 each), DDS (Analog-Device; AD9858, $400 each), 1-Watt power amplifier ($100), pre-amplifier ($50), RCVR (AD8343, $4 each). TRANS (AD834, $20 each, and AD8343) and USB (FT2232D, $10 each). Indicated in the parentheses is the cost of the main electronic component used. Others periphery components such as pin connectors (e.g., SMA), capacitors, rf-switches, rf-transformers and rf-filters cost less than $10 each.

The MRR system 100 may be adaptable to operate in various modes to detect NMR-active nuclei such as proton, fluorine, phosphorus and carbon. The magnetic field used in each mode in which the MRR system 100 operates depends on which nuclei are to be detected. Depending on the mode of operation, the MRR system 100 can operate at a magnetic field of between approximately 0.1 and 3 Tesla (T) which can correspond to between approximately 1 and 150 MHz. For instance, when the MRR system 100 is operating in a proton NMR mode, the magnetic field is approximately 0.76 T which corresponds to approximately 31.9 MHz for proton NMR frequency.

The MRR system 100 can be controlled by the FPGA-based rf spectrometer which comprises the pulse programmer and the second DDS. As compared to CMOS technology, FPGA provides the advantages of re-programmability. The FPGA-based rf spectrometer may, for example, be programmable using tools and software provided by vendors such as Altera Corporation of San Jose, Calif., U.S.A. and Xilinx, Inc. of San Jose, Calif., U.S.A. In an exemplary embodiment, the FPGA chip can include the EP3C80F780C8N, Cyclone III (Altera) embedded on a breadboard (ACM-202-80C8, HumanData, Japan). This chip has 81000 logic elements and is capable of producing 3 independent if-outputs, when fully utilized.

The pulse programmer can generate high power excitation rf pulses. The generated rf pulses then pass through the first power amplifier to produce optimized rf-power for a duration of approximately between 1 and 1000 microseconds to excite all the nuclei effectively. The high power rf pulses are transmitted to the rf probe and will be discussed further herein.

In an exemplary operation, power used for liquid state and solid-state NMR is approximately between 0.1 W and 10 W and approximately between 100 W and 1000 W, respectively. A “strong” power amplifier is often indispensable in MRR systems and such “strong” power amplifiers are often bulky, and require high power consumption, thereby posing serious limitation for field work. For example, a novel and lightweight 1-Watt power amplifier can be constructed on a 4 cm by 4 cm printed circuit board. A solenoid type microcoil (inner diameter 700 to 1000 μm, for example, 750, 800, 850, 900 or 950 μm) can be further employed to generate a strong oscillating magnetic field, B₁, and picks up a signal from the free induction decay (FID) or spin-echo. By employing the duplexer, the high power excitation if pulses that are to be transmitted to the rf-probe in the transmission mode can be isolated from the receiver or detection coil 110 during the receiving mode. The FID/spin-echo is then amplified by a pre-amplifier (AMP-75+, Mini Circuits, USA) with a gain of 20 dB and noise figure of 2.83, and finally filtered by appropriate low pass filter before going into the receiver circuit. FID is the observable NMR signal generated by non-equilibrium nuclear spin magnetization precessing about the static magnetic field (conventionally along z-axis). This non-equilibrium magnetization can be induced, by applying a pulse of resonant radio-frequency close to the Larmor frequency of the nuclear spins. Spin-echo is the refocusing pulse after a single 90-degree inversion followed by inverting them by an 180-degree pulse at resonant.

The magnet system 120 may be portable and light weight (for example, about 60 g) and adaptable to produce a high static field. The magnet system 120 may comprise at least one magnet disposed adjacent to the rf probe. Alternative embodiments include having at least two magnets disposed adjacent to the rf probe. The rf probe can be disposed between the at least two magnets. The magnet system 120 can comprise a permanent magnet and/or an electromagnet. Permanent magnets used in the magnet system 120 may, for example, include Neodymium based magnets.

A detection region of the magnetic resonance relaxometry device can include a volume of less than about 1 μl of the sample for detection. For example, the sample can be provided in sensor, such as a capillary tube or microcapillary tube or a chamber. The sample can be sealed from the ambient environment, reducing exposure to oxygen and other materials that could negatively impact the ability of the magnetic resonance relaxometry device to detect senescent cells.

As described herein, and building on the description of the device described above, a method of detecting senescent cells can include loading a liquid sample including a plurality of cells in a sensor, placing the sensor including the liquid sample within a detection coil of a magnetic resonance relaxometry device, and determining a T₂ value to detect an amount of senescent cells in the liquid sample. Details of determining a T₂ value to detect an amount of senescent cells are described below. For example, the T₂ value can decrease as a number of senescent cells increases. In certain circumstances, determining a T₂ can include measuring a magnetic susceptibility index of the cells.

The current μMRR set up is only suitable for measurement of cell suspension, for example, in a liquid sample. A μMRR set up can be designed to adherent cells on cell culture plates. The devices, systems and methods described herein can be used to develop and improve cell therapies by facilitating removal of senescent cells from cell populations through simplifying the identification process for senescent cells and using that approach in conjunction with cell manipulation technologies.

In certain examples, the amount of senescent cells in the liquid sample can be determined relative to a reference sample. The device can be calibrated in order to rapidly and reliably determine the amount of senescent cells in the liquid sample. Alternatively, device can compare two samples to determine a relative amount of senescent cells in the liquid sample. For example, two samples can be distinguished as having higher and lower relative concentrations of senescent cells.

In certain examples, the amount of senescent cells in the liquid sample can be proportional to a ferritin concentration in the liquid sample. As discussed below, an unexpected connection between an amount of ferritin in a liquid sample and an amount of senescent cells in a liquid sample is one feature that facilitates the use of a magnetic resonance relaxometry device to monitor, measure or determine the presence of senescent cells in a sample.

One way of determining a T₂ value to detect an amount of senescent cells can include quantifying an amount of Fe³⁺ in the liquid sample. In certain circumstances, determining a T₂ can include measuring a content of other paramagnetic or ferromagnetic ions of the cells, for example, copper, specifically copper ions. Other paramagnetic or ferromagnetic ions can include cobalt, nickel, or manganese, cobalt, nickel, or manganese ions. The magnetic resonance relaxometry device can include a radio frequency probe that can be configured to monitor proton relaxation behavior that can then been correlated to Fe³⁺. For example, determining the T₁ or T₂ value can include supplying a train of pulses over a period of less than five minutes, less than four minutes, less than three minutes, less than two minutes or less than one minute.

A number of approaches can be taken to improve the accuracy of the detection of senescent cells. For example, determining the T₁ or T₂ value can include obtaining and averaging a plurality of scans. Up to 100 (or more) scans can be averaged. The number of scans can be less than 80, less than 70, or less than 60. More typically, 10 to 50 scans can be averaged. As a minimum, under certain circumstances, 2 scans, 4 scans, 5 scans, 8 scans, 10 scans, 12 scans, 14 scans, 16 scans, 18 scans, 20 scans, 22 scans, 24 scans, 26 scans, 28 scans, or 30 scans can be averaged. In certain circumstances, the T₁ relaxation time can be used in conjunction with T₂ value to determine whether senescent cells are present in the sample.

The devices, systems and methods described herein can be used to monitor or analyze a number of different cell types for senescence. Identifying senescent cells in a population of cells can allow the population to be refined or purified to remove the senescent cells, improving the productivity of the population of cells in desired uses such as therapies. For example, the cells can include stem cells or progenitor cells. For example, the stem cells or progenitor cells can include mesenchymal stromal cells (MSCs), hematopoietic stem cells (HSCs), or induced pluripotent stem cells (iPSCs). For example, mesenchymal stromal cells (MSC) can then be used more effectively in desired cell therapies.

The system described above can be used to improve efficacy of mesenchymal stromal cells for patients. The method of improving efficacy can include detecting senescent cells in a sample as described above, and concentrating cells in the sample to reduce a number of the senescent cells in the sample. The cell separation device can be a cell separation system including a membrane, a sequestration agent to remove senescent cells, or a microfluidic device. For example, concentrating cells can include passing the sample through a cell separation device.

The cell separation device, for example a microfluidic spiral path device, can be used to separate senescent cells from non-senescent cells in the sample. The cell separation device can include a microfluidic cell sorter, an inertial focusing device, a microfluidic filtration device, a centrifugal flow device, a deterministic lateral displacement (DLD) chip, a tangential flow microfiltration device, or combinations thereof. The cell separation device can remove senescent cells based on monitoring of senescent cells. For example, the cell separation device can removes senescent cells from a cell batch (for example, a cell batch from which the liquid sample was taken) based on detecting the amount of senescent cells.

Referring to FIG. 1B, a system described herein can include magnetic resonance relaxometry device 200 and cell separation device 240. For example, a system for detecting senescent cells can include a magnetic resonance relaxometry device configured to detect an amount of senescent cells in a liquid sample, and a cell separation device for reducing the amount of senescent cells in the liquid sample based on output from the magnetic resonance relaxometry device.

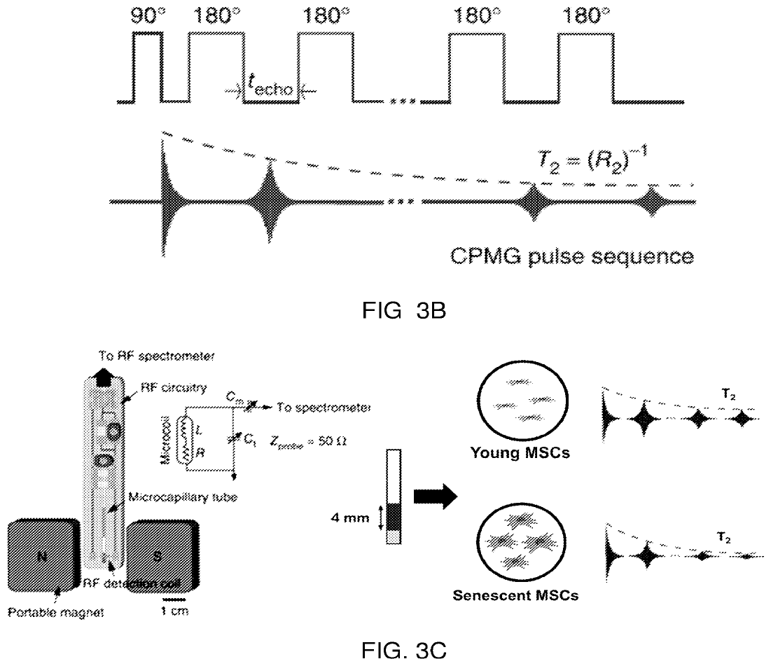

Non-invasive and rapid detection of senescent MSCs using magnetic resonance relaxometry is described. Referring to FIG. 3A, is has been surprisingly discovered that iron ion chemistry in normal cells and senescent cells are different. Normal cells are maintaining iron homeostasis, mediated by numerous iron transporters and iron-binding proteins. Senescent cells are correlated with accumulation of paramagnetic Fe³⁺ (inactive form of iron). For example, a normal cell experiences Fe²⁺ and Fe³⁺ equilibrium and exchange, whereas senescent cells accumulate Fe³⁺.

Referring to FIG. 3B, the CPMG (Carr-Purcell-Meiboom-Gill) pulse sequence for measuring the T₂ values can work efficiently in inhomogeneous magnetic fields produced by permanent magnet of MRR. A train of radiofrequency pulses are applied to the proton nuclei at the resonance frequency of 21.65 MHz with the inter echo time interval t echo and it is repeated for thousands of echoes until relaxes over time. This decayed height of echoes over time is called transverse relaxation time T₂.

Referring to FIG. 3C, an MRR system can consists of portable permanent magnet which provide strong magnetic field. A home built radiofrequency (RF) detection probe is connected to RF spectrometer. The MSC sample (young/proliferating/senescent MSCs) in micro capillary tube can be placed in RF detection coil for T₂ measurements. The micro capillary tube containing the 4 μl of MSC sample in the 4 mm detection range of the RF detection coil. Typical cell number required is 60,000 MSCs within the detection volume. The light region is the crystoseal to seal the micro capillary tube. The right side is showing the 1H spin-spin relaxation time T₂ of young and senescent MSCs in which decreased T₂ in senescent MSCs than Young MSCs.

### Results

### MRR Assay Confirms Higher Senescence Level in Large MSCs Enriched by the Size Sorting Using the Spiral Microfluidic Device

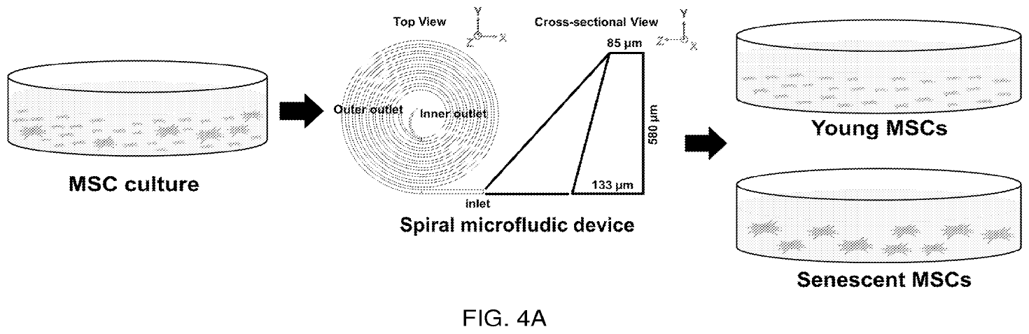

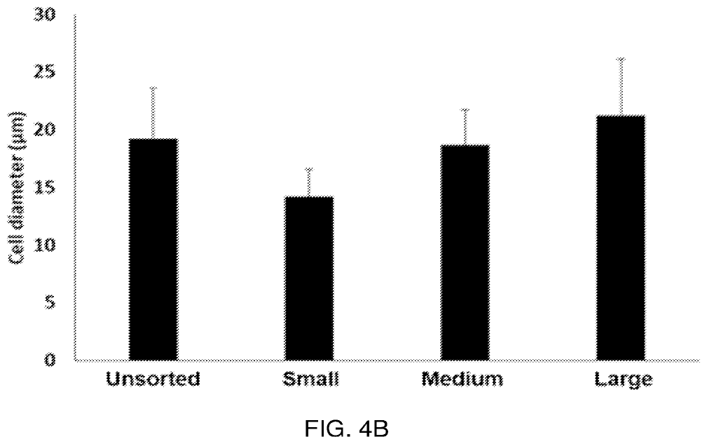

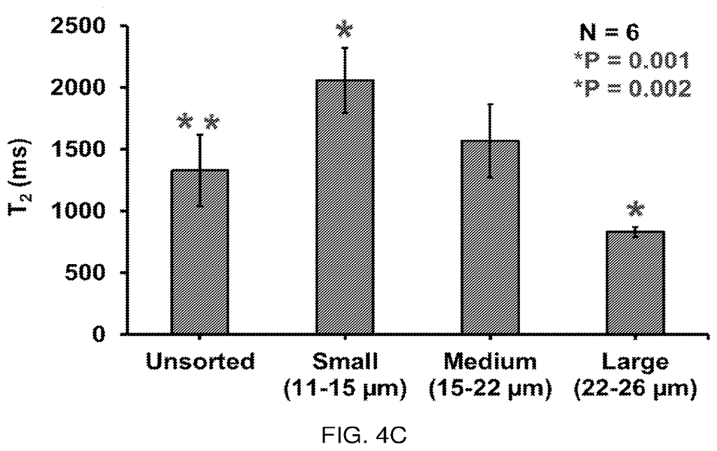

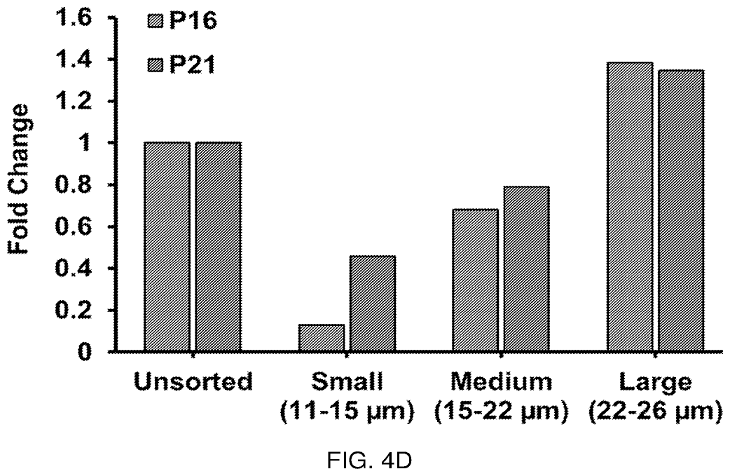

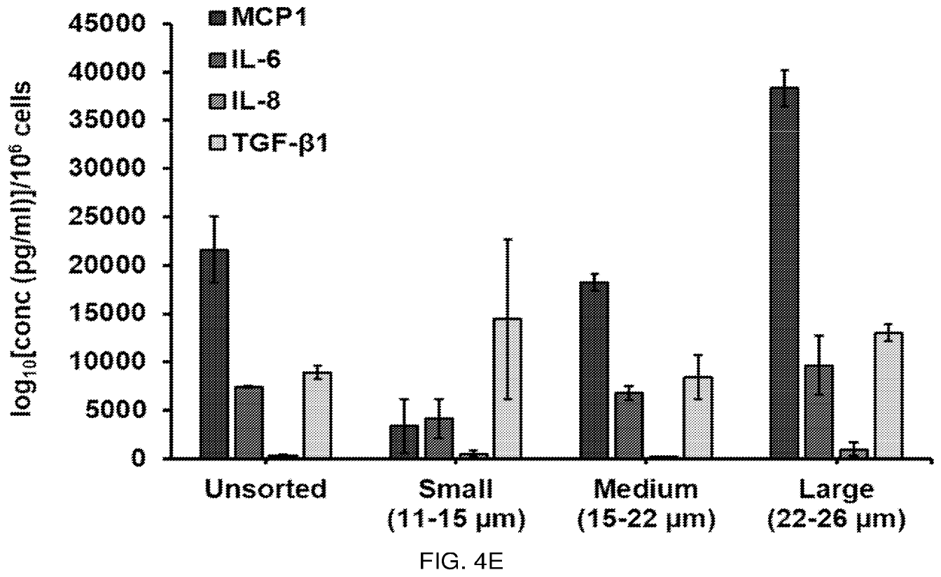

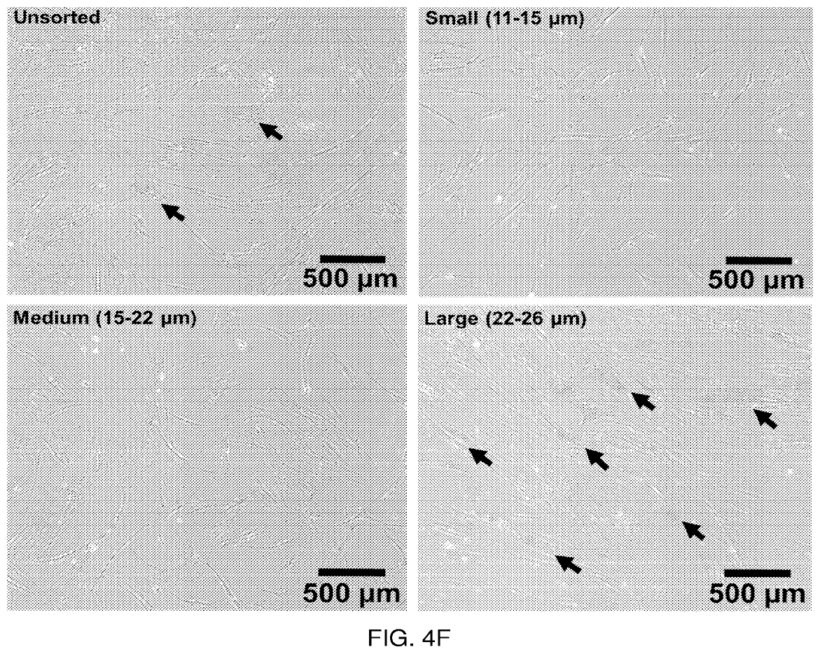

The formation of senescent in MSC culture often diminishes cells' therapeutic efficacy in preclinical and clinical trials. Previously, cell size was identified as a non-invasive marker for distinguishing MSC phenotypes (Refs. 48 and 49), along with other biophysical attributes. Smaller MSCs are generally multipotent MSCs, whereas larger MSCs tend to display limited growth and differentiate more prominently toward the bone lineage. It was found that Large MSCs sorted by inertial microfluidic devices displayed senescent phenotypes with limited growth potential and longer doubling times compared to smaller MSCs (Ref. 50) (FIGS. 4A-4F). MRR assay which show large MSCs are more senescent based on size sorting using spiral microfluidic device. Using the same microfluidic device, MSCs were sorted into three size groups (Unsorted, Small (11-15 μm), Medium (15-22 μm, and Large (22-26 μm), see FIGS. 4A and 4B). The size-sorted cells were then analyzed using μMRR (FIG. 4C), where MSCs were added to the micro-capillary tube and placed inside the radio frequency (RF) detection coil of the uMRR machine for T₂ measurements. MRR analysis of the sorted MSCs showed decreasing T₂ value from Small to Large cells (FIG. 4C), indicating water proton nuclear relaxation, which is ˜2000 ms in pure water, is significantly reduced due to the paramagnetic iron (Fe³⁺) impurity in senescent MSCs. In addition, the expression levels of senescence-associated markers p16 and p21 (measured by qPCR) were elevated in the Large cells compared to Medium and Small groups (FIG. 4D). Furthermore, β-gal staining (FIG. 4E) strongly correlated with both MRR and qPCR results. Higher intensity of β-gal staining was observed in Large and Unsorted group cells, in line with the lower T₂ values measured by uMRR. These results support previous reports of Fe³⁺ storage in senescent cell (Ref. 36), because faster T₂ relaxation is due to the increase of paramagnetic Fe³⁺ content in the cells. The secretion of molecules associated with senescence-associated secretory phenotype (SASP) was also measured for the unsorted and size-sorted MSC subpopulations (Small, Medium, and Large) (FIG. 4D). Higher expression of key secreted markers associated with SASP was found in larger cells MSCs. See, for example, Lu Yin, Zheng Yang, et al., Biomaterials, 240, 119881 (2020), which is incorporated by reference in its entirety.

MRR-Based Senesnesce Measurements are Correlated with Altered Multi-Lineage Differentiation Potential of MSCs from Different Donors

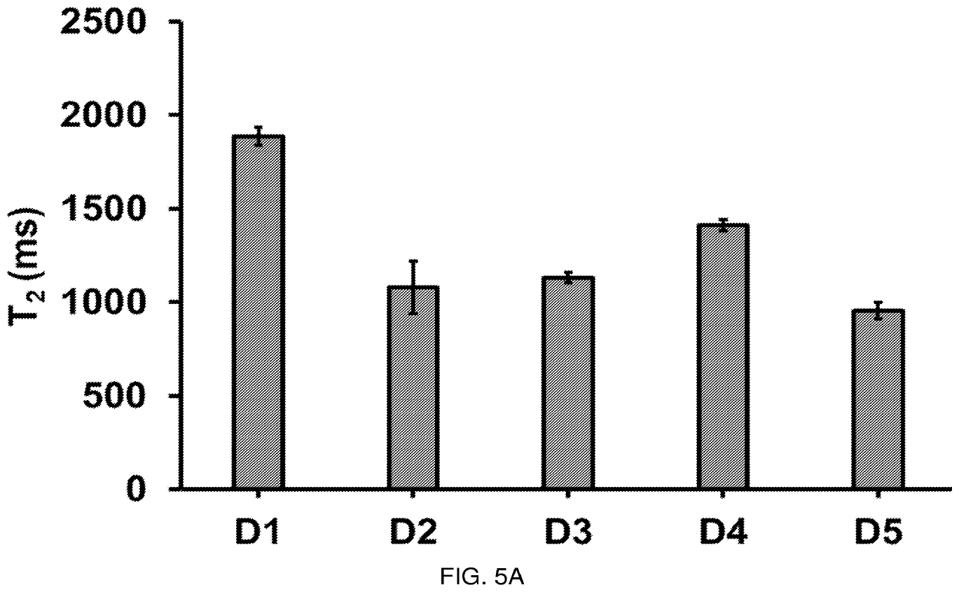

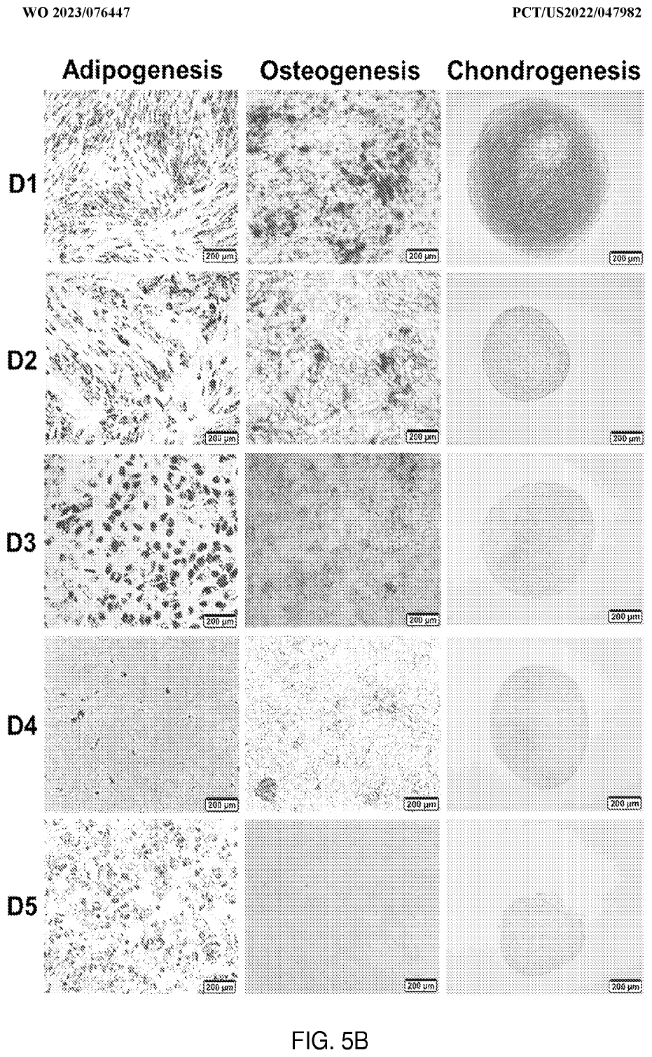

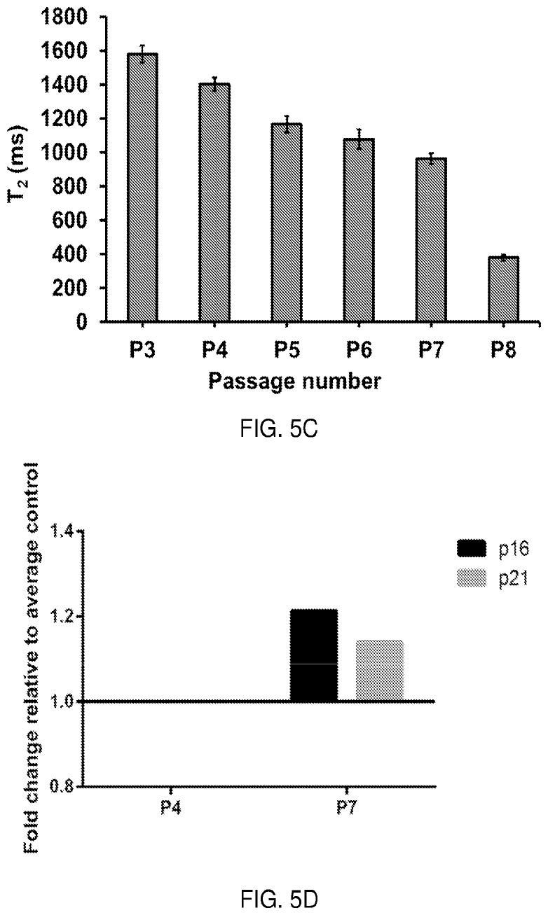

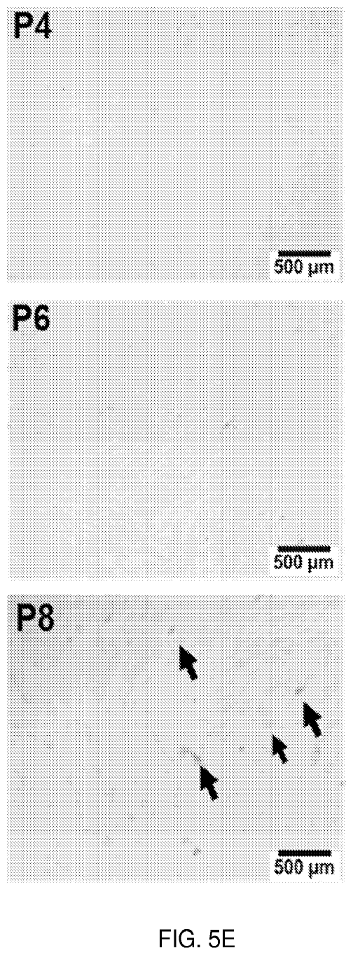

MRR analysis measures altered multi-lineage differentiation in MSCs from different passages and donors. A critical therapeutic quality of MSCs is their ability to undergo multi-lineage differentiation into cell lineages such as adipocytes, chondrocytes, and osteoblasts. The MSCs from different donors vary in their multi-lineage differentiation potential, which has been a critical and poorly understood limitation in achieving consistent quality control of MSCs for therapy. Here, MSCs were analyzed from five different donors (D1 to D5; See Methods for description of donors) cultured in vitro to passage 3. For MRR analysis, a normalized cell number of 6×10⁴ was used for each T₂ experiment. FIG. 5A shows the T₂ profile of MSCs from different donors. Multi-lineage differentiation potential of MSCs from the corresponding donors (D1 to D5) was measured by staining (FIG. 5B). By analyzing the differentiation potential, it was observed that D1 MSCs were fully capable of multi-lineage differentiation potential and D5 had the most limited potential. T₂ values of MSCs from each donor correlated with overall differentiation potential, as the highest T₂ value was observed for MSCs from D1, whereas the lowest T₂ value was measured for MSCs from D5. Hence, the lower T₂ values corresponded to a decline in multi-lineage differentiation potential. Decreased multi-lineage differentiation potential in MSCs from certain donors may be affected by the presence of higher proportion of senescent cells in the population, and this could also influence cell proliferation.

### MRR Analysis Shows an Increase in Cellular Senescence Upon Serial Passages of MSCs

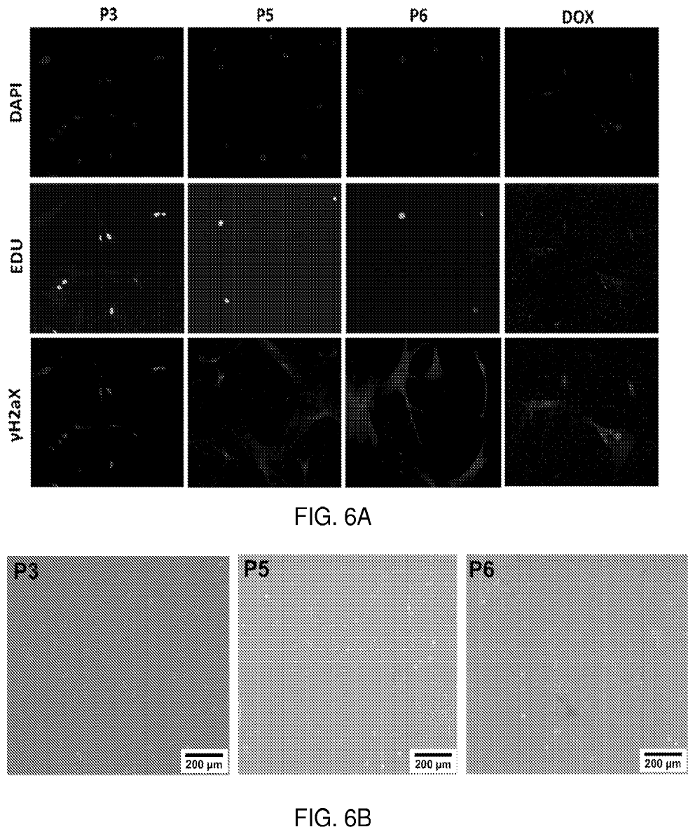

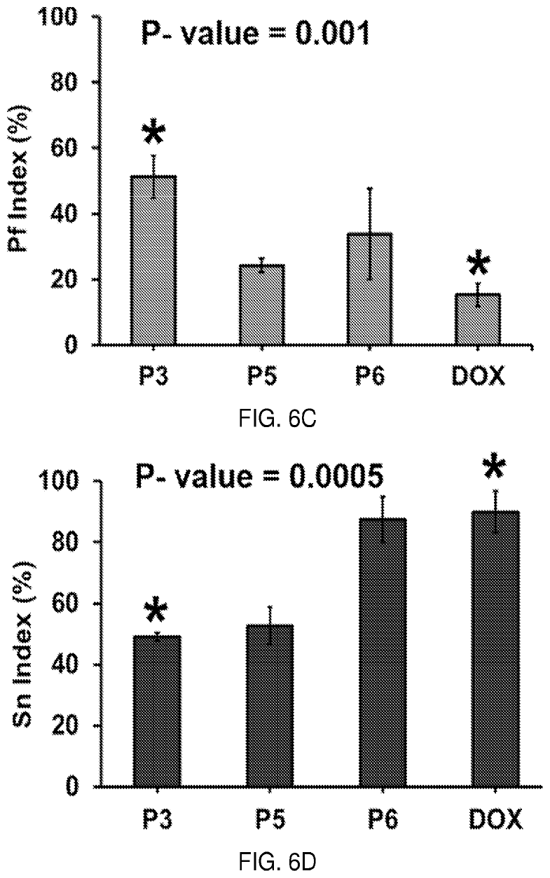

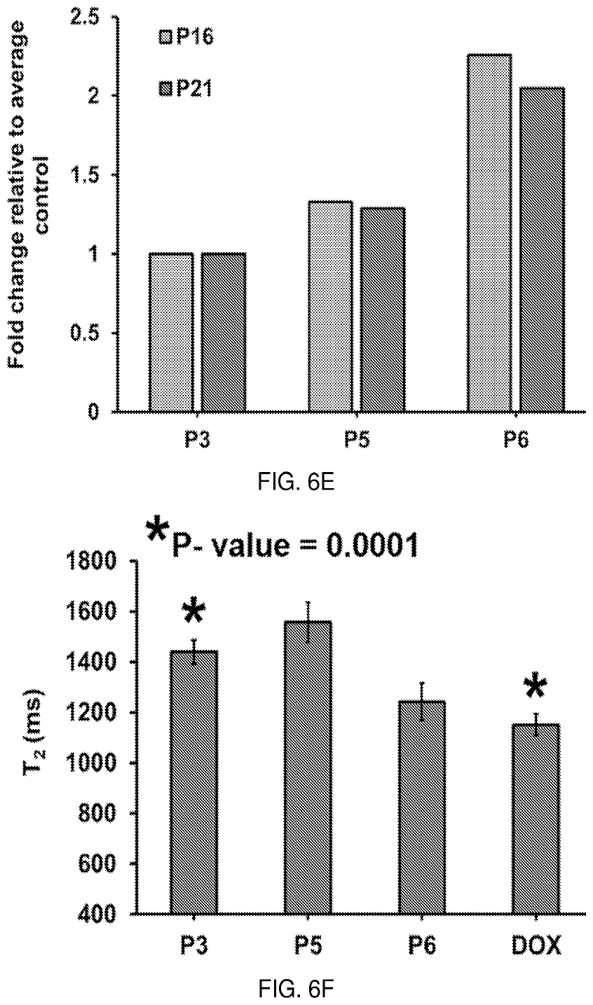

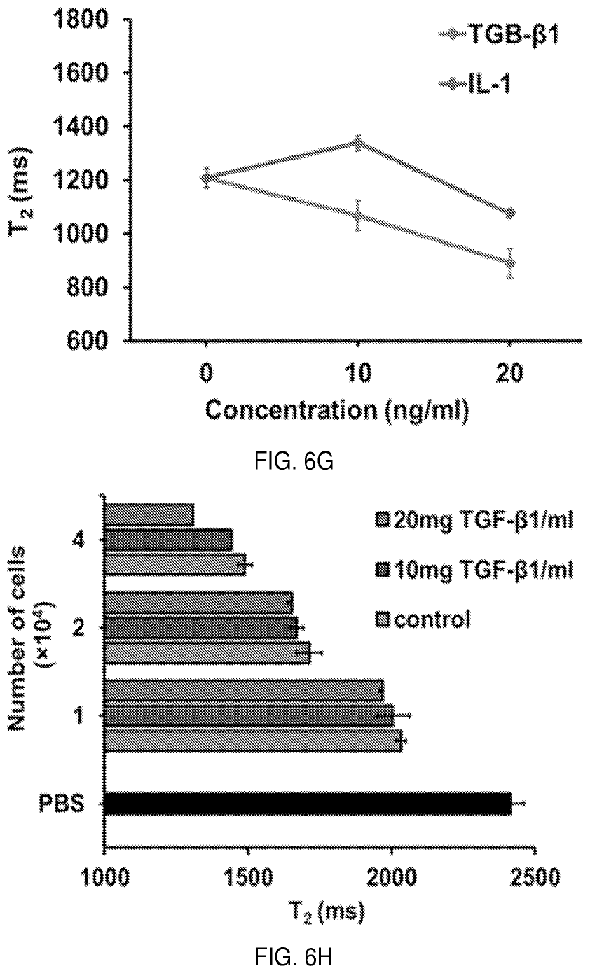

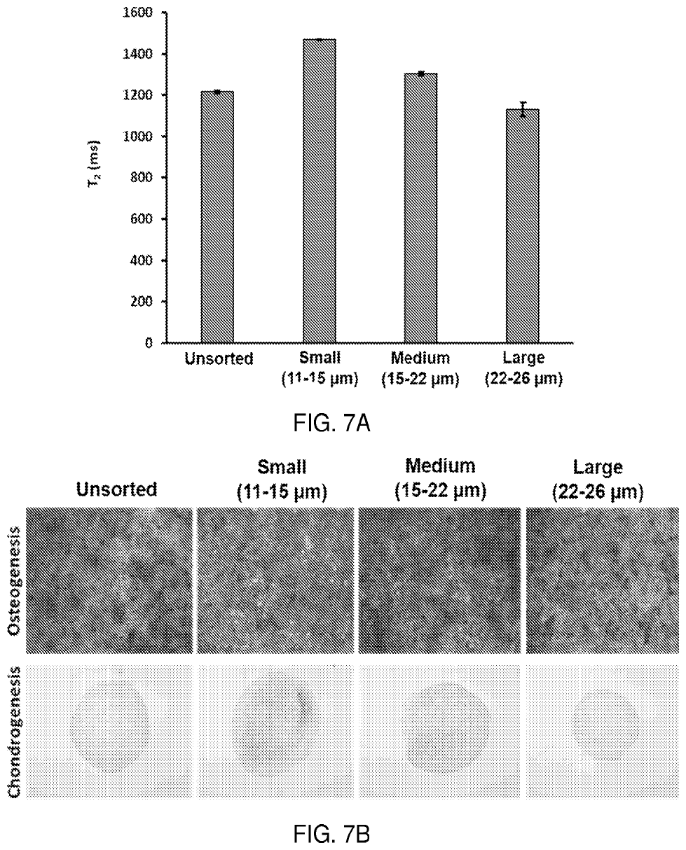

It is possible to correlate MRR with standard assays. During in vitro culture of MSCs, cellular senescence tends to increase during serial passaging. Senescent cells can affect the proliferation of MSCs, and differentiation potential and hence the quality of cells for their clinical use. Therefore, it is important to rapidly identify senescent cells, and potentially remove them from each passage of MSC culture. (Ref. 50) Donor 1 MSCs were next analyzed from serial passages P3 to P8 under in vitro culture conditions. Using uMRR, T₂ values of normalized concentration of MSCs from each passage are shown in FIG. 6G. The highest T₂ value for MSCs were observed in the early passage, whereas these levels progressively decreased at later passages. MSCs from the later passage, where the proportion of senescent cells was greater based on the intensity and number of cells displaying β-gal staining showed the lowest T₂ value (FIG. 6H). Similarly, qPCR of the cell cycle inhibitor markers p16 and p21 showed highest expression in cells from late passages as compared to early passages (FIG. 6E).

### Senescence Detection by Immunofluorescent Assay and MRR

Proliferation and senescence were measured simultaneously in order to correlate cell state directly with the MRR T₂ values. MSCs from the same donor were serially passaged to passage 3, passage 5, and passage 6. As a positive control for senescence cells, cells from passage 3 were also treated with doxorubicin (DOX), a chemotherapy agent that produces DNA damage and reactive oxygen species to induce senescence in tumor cells. To measure DNA synthesis, EdU (5-ethynyl-2′-deoxyuridine) was used as an indicator of active cell proliferation (shown in green). In contrast, senescent cells are known to accumulate γ-H2AX foci (indicated in red). The proliferation (Pf) index and senescence (Sn) index were calculated from these images and plotted respectively (FIG. 6C and FIG. 6D). MRR experiments of cells from all passages and DOX-treated cells were also performed, and T₂ values are shown for each condition (FIG. 6F). From staining images and Pf/Sn index graphs, more proliferating cells (EDU) were present in P3. Pf gradually decreased with the later passages (P5 and P6) and was the lowest in DOX-treated cells. The Sn Index showed the opposite trend where more senescent cells (γ-H2aX) were observed in P6 and in DOX-treated cells, compared to P3 and P5. These results were in strong agreement with the T₂ measurements (FIG. 6F), supporting the MRR assay as a robust method to detect senescent cells and non-invasively. In addition, there was a greater proportion of β-gal-stained cells in the later passage (P6), consistent with the T₂ results from respective passages. Similarly, qPCR in which higher expression of p16 and p21 were observed in MSCs from P6 (FIG. 6E) supported the uMRR measurements.

### Induced Senescence by Treatment of Cytokines

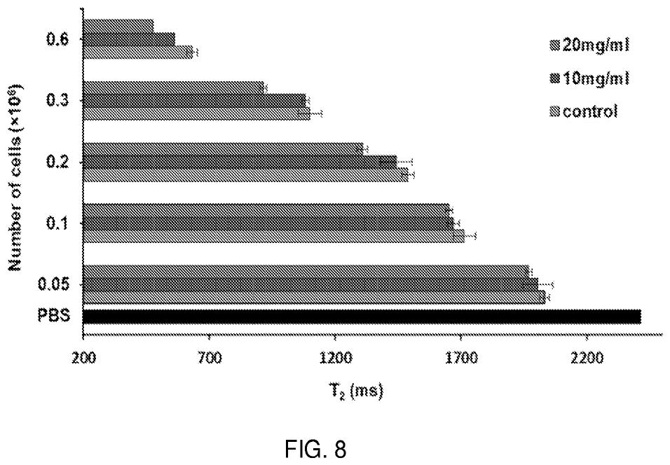

Cytokines such as Transforming Growth Factor-β1 (TGFβ1) and interleukin-1 (IL-1) induce senescence in MSCs. TGFβ1 is a TGF family member that controls numerous cellular functions, including proliferation and cell death. (Refs. 51 and 52) IL-1 is a proinflammatory cytokine whose expression is associated with SASP. (Ref. 53) Hence, MSCs were treated with different concentrations (10 ng and 20 ng/ml) of TGFβ1 and IL-1 for and performed measurements using the mMRR approach. T₂ values of TGFβ1 and IL-1 treated cells decreased with increasing cytokine concentrations. The senescent cells are more in TGF-β1 treated cells than IL-1 treated cells (FIG. 8), which is also confirmed here by its decreased T₂ values than IL-1 treated cells.

Since MSCs donors show differences in proliferative ability, it is important to find the limit of detection (LOD) of MRR assay in terms of the cell number. For all uMRR experiments, the normalized cell number was 6×10⁴ in 4 μl volume in the microcapillary tube. Here, the LOD was analyzed by decreasing the concentration of cells even lower to 4 ×10⁴, 2×10⁴, and 1×10⁴ cells for uMRR detection as shown in the FIG. 6H and FIG. 8. The MSCs are treated with TGFβ1 at different concentrations (10 and 20 mg/ml) to induce senescence, and these cells are collected for MRR detection. The batches of 1, 2, and 4 (×10⁴ cells) in 4 μl detection volume of TGF-β1 treated and untreated MSCs were analyzed by MRR. The T₂ value of PBS is also compared with T₂ value of MSCs (treated and untreated). This analysis shows a significant difference in T₂ value of senescent MSCs when compared to the T₂ value of untreated MSCs and PBS even at the lowest concentration of 1×10⁴ cells. Together, the data described herein suggest that uMRR can be used to non-invasively measure the presence of senescent cells in a heterogenous culture, which may be employed to improve bulk culture of clinical grade cell therapy products such as MSCs.

A new method for quantifying senescent cells in vitro has been established, which is rapid, non-invasive, requires a small number of cells (<10⁵), and does not require any reagents or sample preparation steps. Direct Fe³⁺ quantification by uMRR provides well-correlated results with conventional end-point and time-intensive assays typically used to measure senescent cells such as qPCR, Luminex assay, and β-galactosidase staining. Since paramagnetic Fe³⁺ (but not diamagnetic Fe²⁺) increases the magnetic susceptibility, which stimulates proton nuclear relaxation of water molecules in the cells, this measurement is specifically quantifying the Fe³⁺ content of cellular iron. Previously, intracellular Fe²⁺ (labile iron) was measured by colorimetric and other assays (Ref. 54), yet reliable and quantitative detection of Fe²⁺ has generally been challenging (Refs. 55 and 56), presumably due to the reactive nature of Fe²⁺. For Fe³⁺ quantification, ferritin is often used as a surrogate marker (Ref. 57), which is a large (˜480 kDa) protein complex that can accommodate up to 4500 atoms of Fe³⁺ iron, but ferritin concentration may not correlate well with the Fe³⁺ amount stored in the cell. ICP-MS quantification (Ref. 58) of total iron concentration measurement is the current standard for iron quantification, but this may not correctly reflect the stored Fe³⁺, which is more closely correlated to cellular senescence.

In contrast to the conventional iron quantification methods, MRR is uniquely positioned to advance iron biology in general. μMRR allows completely non-invasive phenotyping of senescent cells, allowing downstream biological and functional measurements of other indicators on the same cells. This ability will enable us to build strong and direct correlations between MRR and other biochemical and functional measurements. This approach is potentially transformative for the field, especially given the lack of consensus cell surface markers specific for senescent cells.

In addition, the ‘iron-imaging’ modality utilized here is already widely used in T₁ and T₂ weighted imaging of MRI (Refs. 59-62). Therefore, any insights from MRR quantification of cellular senescence could readily be compared to in vivo pathology. Given the resolution of modern MRI approaches below 100 μm (Ref. 63), non-invasive detection of senescent cells in live tissue may be feasible in the future. Most importantly, the ability to quantify the degree of senescence in MSCs would be uniquely enabling for therapeutic application of MSCs, where the donor-dependent quality variations of MSCs, which are largely due to different degree of senescence, is a significant unsolved bottleneck. μMRR is envisioned as a rapid, non-invasive quality screening tool for MSCs, during the culture-based production process.

In this study, the multi-lineage differentiation potential of MSCs from different donors also correlates well with MRR results, with the highest T₂ value measured from MSCs having robust multi-lineage differentiation potential. This technology will be instrumental for screening MSCs for the highest growth and differentiation potential in a patient specific manner in real time. One can also adapt this new bioengineering modality for optimizing biomanufacturing of MSC or other cell therapy products. For example, MRR can be used to monitor cell cultures in real time in a way that would allow removal of senescent cells using microfluidic sorting (Ref. 50). Thus, this approach could overcome a major bottleneck in the field to produce higher grade MSCs and cell products for therapeutic applications.

### Methods and Materials

### MSC Culture

Bone marrow-derived mesenchymal stem cells (MSC) were purchased from Lonza Pte. Ltd. and Rooster Bio Inc. The cells were expanded in tissue culture plate (TCP) at an initial cell density of 1500 cells/cm² in low glucose Dulbecco's Modified Eagle Medium (DMEM) supplemented with 10% fetal bovine serum (FBS), 1% GlutaMAX, and 1% Penicillin/Streptomycin (Thermo Fisher Scientific, Singapore) at 37° C. in 5% CO₂ atmosphere. The medium was changed every two days, and the cells were harvested at 80% confluency for further experiments or subcultures. During the cells harvesting, the cells were incubated with 0.25% Trypsin-EDTA (Thermo Fisher Scientific, Singapore) for 3 minutes. The cell number was calculated using a disposable hemocytometer (INCYTO, Korea) with the trypan blue exclusion method. MSCs at passage 3 (P3) to P5 were used for further experiment unless otherwise stated. MSC senescence induction was carried out by treatment with culture media containing 5, 10, or 20 ng/ml of TGF-β1 for three days. For doxorubicin (DOX) treatment, cells were seeded at 1500 cells/cm² and incubated with 1 μM doxorubicin (Sigma) for 24 hours at 37° C. Subsequently, the medium was removed, and a fresh medium was added to the cells to incubate for 24 hours before analysis.

MSC Sorting with Inertial Spiral Microchannel Device

The inertial spiral microchannel device was designed and fabricated in the same way as previously described. (Ref. 50) The device has 8 loops with a radius decreasing from 12 mm to 4 mm and a trapezoidal cross-section with 580 μm width, 85 μm inner, and 133 μm outer height. It has one inlet for the introduction of the cell suspension to be sorted and two outlets for the collection of sorted cells. The design was carved on a micro-milled aluminum mold (Whits Technologies Inc., Singapore), and the device was cast from polydimethylsiloxane (PDMS) with a ratio of 10:1 base and curing agent mixture (Sylgard 184, Dow Corning Inc., USA).

Before sorting, MSC was resuspended in culture media at 1-2 million cells/ml and loaded into syringes (Thermo Fisher Scientific, Singapore) that were connected to Tygon tubing (Spectra Teknik Pte. Ltd., Singapore). The tubing was inserted into the inlet of the device, and two separates tubing was inserted at the outlets for the collection of sorted cells. A syringe pump (PHD2000, Harvard Apparatus Inc., USA) was used to control the flow rate of cell suspension in the device. Two serials sorting was performed to separate MSC into three populations of different sizes. The first sorting was carried out at 3.5 ml/min, and the cells collected at the inner outlet are the largest subpopulation (22-26 μm). The cells collected at the inner outlet were subjected to second sorting at 1.5 ml/min, and the cells collected at the inner and outer outlets are medium size subpopulation (15-22 μm) and smallest subpopulation (11-15 μm), respectively. The unsorted MSC was used as a control in this experiment. Real-time visualization of the separation within the inertial spiral microchannel device was achieved by an inverted microscope (IX71, Olympus Co., Japan) equipped with a high-speed CCD camera (Phantom v9, Vision Research Inc., USA).

### MRR Measurement

MRR consists of a portable permanent magnet (Metrolab Instruments, Plan-les-Ouates, Switzerland) with B0=0.5 T and a bench-top type NMR console (Kea Magritek, Wellington, New Zealand). 1H MRR measurements were performed at the resonance frequency of 21.65 MHz inside the magnet. A single resonance proton MRR probe with a detection micro coil of 900-μm inner diameter was used for accommodating the MRR samples into the microcapillary tubes (o.d.: 1,500 μm, i.d.: 950 μm) (22-260-950, Fisherbrand, Waltham, MA, USA). In MRR probe, the electronic parts and coil were mounted on the single printed circuit board (FIG. 3C). (Ref. 40) All the experiments were performed at 26.3° C. inside the magnet which is maintained by a temperature controller (RS component, UK).

For all MRR experiments, the normalized concentration (3×10⁵ cells) of MSCs have used unless otherwise stated. The MSCs samples were spun down at 300 g for 5 minutes and aspirate the supernatant. The pellet is suspended in 20 μl of PBS and filled at a 4 mm length of micro-capillary tube. The micro-capillary tube was sealed with crystoseal and mounted it in to the coil for MRR measurements. Proton transverse relaxation rates T₂ were measured by standard Carr-Purcell-Meiboom-Gill (CPMG) pulse programme (Refs. 64 and 65) (FIG. 3B). The transmitter power output was maintained at 12.5 mW for a single 90° pulse of pulse length 16 us for all the T₂ measurements. The CPMG train of pulses with inter echo time of 60 us with 4000 echoes was used for all experiments. A recycle delay of 2 s, which is sufficient to allow all the spins to return to thermal equilibrium, was used. 24 scans were performed for all experiments for signal averaging.

Real Time Polymerase Chain Reaction (qPCR) Analysis

Total RNA was extracted with the RNeasy Mini Kit (Qiagen, USA) following the manufacturer's protocol. The concentration of RNA was determined using a NanoDrop UV-vis Spectrophotometer (NanoDrop Technologies, USA). After which, reverse transcription reaction was carried out with 200 ng total RNA using iScript™ cDNA synthesis kit (Biorad Laboratories, USA). Real-time PCR was performed using SYBR Green system with primers listed in Table 1 ABI 7500 real-time PCR system (Applied Biosystem, USA) was used to performed real-time PCR at 95° C. for 10 mins and 40 cycles of amplification which encompasses denaturation step at 95° C. for 15s and extension step at 60° C. for 1 min. The gene expression level was normalized to glyceraldehyde-3-phosphate dehydrogenase (GAPDH) and calculated using the 2^{-ΔΔCt} formula with reference to the respective control group.

TABLE 1
The primers for Real time PCR

| Primer | Forward sequences | Reverse sequences |
|---|---|---|
| GAPDH | ATGGTGAAGGTCGGAGTGAA | AATGAAGGGGTCATTGATGG |
| P16 | CACCGAATAGTTACGGTCGG | GCACGGGTCGGGTGAGAGTG |
| P21 | TCTTGTACCCTTGTGCCTCG | AGAAGATCAGCCGGCGTTTG |

### Conditioned Media and ELISA

Size-sorted MSCs were seeded in T75 flasks for overnight adherence before media removal the next day. The cells were washed once with PBS and cultured in serum-free media for 48 hours at 37° C., 5% CO₂. The media was then collected and centrifuged at 4500×g for 15 minutes to remove cell debris. The supernatant was collected and concentrated to 500 μl with an Amicon Ultra 15 filter (3 kDa cut-off membrane and stored at −80° C. before analysis. Cell number in each condition was determined to normalize the concentration values of the analytes of interest (MCP-1, IL-6, IL-8, and TGF-β1) that were obtained using Luminex®-based multiplex assays (R&D Systems) according to manufacturer's instructions. All samples were analyzed in technical duplicates and performed for two different donors. The data were obtained with a MAGPIX reader (Millipore), and concentrations were derived from measured mean fluorescence intensities (MFI) using fitted standard curves using 5-parameter logistic regression (SSL5) using the Milliplex Analyst software (Millipore).

### In Vitro Multi-Lineage Differentiation

MSCs were seeded in 24-well plates for overnight adherence before media removal and replaced with specific differentiation media (STEMCELL Technologies) for osteogenesis and adipogenesis, following the manufacturer's protocol. Osteogenesis induction was confirmed with Alizarin Red S staining (ScienCell) for calcium deposits, while adipogenic differentiation was determined with Oil Red O staining (Sigma) for the detection of lipid droplets. For chondrogenesis, MSCs were pelleted at 1×10⁶ cells/pellet in a 15 ml tube at 300×g, 5 minutes. Chondrogenic media (STEMCELL Technologies) was added to this pellet, and media was changed every alternate day for three weeks before glycosaminoglycan staining with Safranin O.

### Senescence Assay

MSCs were seeded on a 6-well plate at an initial cell seeding density of 1500 cells/cm² and cultured for 3-5 days. Senescence β-galactosidase staining kit (Sigma Aldrich) was used to identify the senescent cells according to the manufacturer's protocol. Briefly, the cells were fixed at room temperature for 10 mins and incubated in a staining solution containing X-gal at 37° C. overnight. Cells stained positive with blue precipitate as a result of X-gal substrate cleavage were indicative of senescent cells.

### Immunofluorescence and Confocal Microscopy

For detection of SA-βgal together with another senescence marker (γ-H2AX foci) (Ref. 66) and the proliferation marker, 5-ethynyl-2′-deoxyuridine (Edu) in the cells, the protocol was performed as previously mentioned (Ref. 67). Briefly, the cells were incubated with 10 μM (Edu) (Click-iT® EdU Alexa Fluor® 488 Imaging Kit, Invitrogen) for 24 hours before fixation with 10% neutral buffered formalin (Sigma) and SA-βgal staining using components from the staining kit according to the manufacturer's instructions (#9860, Cell Signaling Technology). Subsequently, Edu labeling was detected following the manufacturer's protocol (Invitrogen).

For immunostaining, cells were washed in PBS and incubated with blocking buffer (PBS containing 0.5% bovine serum albumin) for 1 hour before incubation with a mouse anti-phospho-Histone H2A.X (Ser139) antibody (05-636, Millipore) in blocking buffer for overnight at 4° C. Cells were then incubated with Rhodamine Red™-conjugated secondary antibody (Jackson Immuno Research Laboratories) for 1 hour, washed thrice with PBS, and counterstained with NucBlue (Hoechst 33342)-containing mounting solution (Invitrogen). Images were acquired with an FV1200 Confocal Microscope using a 20 × objective lens (Olympus). Stained SA-βgal cells were also visualized under phase-contrast microscopy.

The following references, many of which are cited herein, are incorporated by reference in their entirety.

### REFERENCES

1. Kuilman, T., Michaloglou, C., Mooi, W. J. & Peeper, D. S. The essence of senescence Thomas. GENES Dev. 24, 2463-2479 (2010).
2. Van Deursen, J. M. The role of senescent cells in ageing. Nature 509, 439-446 (2014).
3. Campisi, J. Aging, Cellular Senescence, and Cancer. Annu Rev Physiol. 75, 685-705 (2013).
4. Hanahan, D. & Weinberg, R. A. Hallmarks of cancer: The next generation1. Hanahan, D. & Weinberg, R. A. Hallmarks of cancer: The next generation. Cell 144, 646-674 (2011). Cell 144, 646-674 (2011).
5. Collado, M., Blasco, M. A. & Serrano, M. Cellular Senescence in Cancer and Aging. Cell 130, 223-233 (2007).
6. Naylor, R. M., Baker, D. J., van Deursen, J. M. Senescent Cells: A Novel Therapeutic Target for Aging and Age-Related Diseases. Clin Pharmacol Ther. 93, 105-116 (2013).
7. McHugh, D. & Gil, J. Senescence and aging: Causes, consequences, and therapeutic avenues. J. Cell Biol. 217, 65-77 (2018).
8. Wagner, W. et al. Replicative senescence of mesenchymal stem cells: A continuous and organized process. PLOS One 3, (2008).
9. Mareschi, K. et al. Expansion of mesenchymal stem cells isolated from pediatric and adult donor bone marrow. J. Cell. Biochem. 97, 744-754 (2006).
10. Sedelnikova, O. A. et al. Senescing human cells and ageing mice accumulate DNA lesions with unrepairable double-strand breaks. Nat. Cell Biol. 6, 168-170 (2004).
11. Von Zglinicki, T. Oxidative stress shortens telomeres. Trends Biochem. Sci. 27, 339-344 (2002).
12. Aird, K. M., Zhang, G., Li, H., Tu, Z., Bitler, B. G., Garipov, A., Wu, H., Wei, Z., Wagner, S. N., Herlyn, M., Zhang, R. Suppression of Nucleotide Metabolism Underlies the Establishment and Maintenance of Oncogene-Induced Senescence. Cell Rep. 3, 1252-1265 (2013).
13. Moiseeva, O., Mallette, F. A., Mukhopadhyay, U. K., Moores, A., Ferbeyre, G. DNA Damage Signaling and p53-dependent Senescence after Prolonged □-Interferon Stimulation. Mol. Biol. Cell 17, 1583-1592 (2006).
14. Bonab, M. M. et al. Aging of mesenchymal stem cell in vitro. BMC Cell Biol. 7, 1-7 (2006).
15. Drela, K., Stanaszek, L., Nowakowski, A., Kuczynska, Z. & Lukomska, B. Experimental strategies of mesenchymal stem cell propagation: Adverse events and potential risk of functional changes. Stem Cells Int. 2019, (2019).
16. Rubin, H. Promise and problems in relating cellular senescence in vitro to aging in vivo. Arch. Gerontol. Geriatr. 34, 275-286 (2002).
17. Banfi, A. et al. Proliferation kinetics and differentiation potential of ex vivo expanded human bone marrow stromal cells: Implications for their use in cell therapy. Exp. Hematol. 28, 707-715 (2000).
18. Schellenberg, A. et al. Replicative senescence of mesenchymal stem cells causes DNA-methylation changes which correlate with repressive histone marks. Aging (Albany. NY). 3, 873-888 (2011).
19. Zhou, S. et al. Age-related intrinsic changes in human bone-marrow-derived mesenchymal stem cells and their differentiation to osteoblasts. Aging Cell 7, 335-343 (2008).
20. Li, Y. et al. Senescence of mesenchymal stem cells (Review). Int. J. Mol. Med. 39, 775-782 (2017).
21. Noer, A., Boquest, A. C. & Collas, P. Dynamics of adipogenic promoter DNA methylation during clonal culture of human adipose stem cells to senescence. BMC Cell Biol. 8, 1-11 (2007).
22. Liang, H. et al. Increased expression of pigment epithelium-derived factor in aged mesenchymal stem cells impairs their therapeutic efficacy for attenuating myocardial infarction injury. Eur. Heart J. 34, 1681-1690 (2013).
23. Campioni, D. et al. A decreased positivity for CD90 on human mesenchymal stromal cells (MSCs) is associated with a loss of immunosuppressive activity by MSCs. Cytom. Part B—Clin. Cytom. 76, 225-230 (2009).
24. Dimri, G. P. et al. A biomarker that identifies senescent human cells in culture and in aging skin in vivo. Proc. Natl. Acad. Sci. U.S.A 92, 9363-9367 (1995).
25. Schellenberg, A., Hemeda, H. & Wagner, W. Tracking of Replicative Senescence in Mesenchymal Stem Cells by Colony-Forming Unit Frequency. Methods Mol Biol 976, 143-154 (2013).
26. Penfornis, P. & Pochampally, R. Colony forming unit assays. Methods Mol. Biol. 1416, 159-169 (2016).
27. Cheng, H. et al. Replicative senescence of human bone marrow and umbilical cord derived mesenchymal stem cells and their differentiation to adipocytes and osteoblasts. Mol. Biol. Rep. 38, 5161-5168 (2011).
28. Debacq-Chainiaux, F., Erusalimsky, J. D., Campisi, J. & Toussaint, O. Protocols to detect senescence-associated beta-galactosidase (SA-βgal) activity, a biomarker of senescent cells in culture and in vivo. Nat. Protoc. 4, 1798-1806 (2009).
29. Fairweather-Tait, S. J., Wawer, A. A., Gillings, R., Jennings, A. & Myint, P. K. Iron status in the elderly. Mech. Ageing Dev. 136-137, 22-28 (2014).
30. Xu, J., Jia, Z., Knutson, M. D. & Leeuwenburgh, C. Impaired iron status in aging research. Int. J. Mol. Sci. 13, 2368-2386 (2012).
31. Puxeddu, E. et al. Iron laden macrophages in idiopathic pulmonary fibrosis: The telltale of occult alveolar hemorrhage? Pulm. Pharmacol. Ther. 28, 35-40 (2014).
32. Ogilvie-Harris, D. J. & Fornaiser, V. L. Synovial iron deposition in osteoarthritis and rheumatoid arthritis. J. Rheumatol. 7, 30-36 (1980).
33. Morris, C. J., Blake, D. R., Wainright, A. C. & Steven, M. M. Relationship between iron deposits and tissue damage in the synovium: An ultrastructural study. Ann. Rheum. Dis. 45, 21-26 (1986).
34. Chen, X., Yu, C., Kang, R. & Tang, D. Iron Metabolism in Ferroptosis. Front. Cell Dev. Biol. 8, 1-14 (2020).
35. Zhou, R. P. et al. Novel insights into ferroptosis: Implications for age-related diseases. Theranostics 10, 11976-11997 (2020).
36. Masaldan, S. et al. Iron accumulation in senescent cells is coupled with impaired ferritinophagy and inhibition of ferroptosis. Redox Biol. 14, 100-115 (2018).
37. Mancias, J. D., Wang, X., Gygi, S. P., Harper, J. W., Kimmelman, A. C. Quantitative proteomics identifies NCOA4 as the cargo receptor mediating ferritinophagy. Nature 509, 105-109 (2014).
38. Dixon, S. J. et al. Ferroptosis: An iron-dependent form of nonapoptotic cell death. Cell 149, 1060-1072 (2012).
39. Gao, M. et al. Ferroptosis is an autophagic cell death process. Cell Res. 26, 1021-1032 (2016).
40. Peng, W. K. et al. Micromagnetic resonance relaxometry for rapid label-free malaria diagnosis. Nat. Med. 20, 1069-1073 (2014).
41. Thamarath, S. S., Xiong, A., Lin, P. H., Preiser, P. R. & Han, J. Enhancing the sensitivity of micro magnetic resonance relaxometry detection of low parasitemia Plasmodium falciparum in human blood. Sci. Rep. 9, 1-9 (2019).
42. Peng, W. K., Chen, L., Boehm, B. O., Han, J. & Loh, T. P. Molecular phenotyping of oxidative stress in diabetes mellitus with point-of-care NMR system. npj Aging Mech. Dis. 6, 1-12 (2020).
43. Castro, C. M. et al. Miniaturized Nuclear Magnetic Resonance Platform for Detection and Profiling of Circulating Tumor Cells. Lab Chip. 14, 14-23.
44. Lee, H., Sun, E., Ham, D. & Weissleder, R. Chip-NMR biosensor for detection and molecular analysis of cells Hakho. Nat Med. 14, 869-874 (2008).
45. Haun, J. B., Devaraj, N. K., Hilderbrand, S. A., Lee, H. & Weissleder, R. Bioorthogonal chemistry amplifies nanoparticle binding and enhances the sensitivity of cell detection. Nat Nanotechnol. 5, 660-665 (2010).
46. Liong, M. et al. Magnetic barcode assay for genetic detection of pathogens. Nat. Commun. 4, (2013).
47. Issadore, D. et al. Miniature magnetic resonance system for point-of-care diagnostics. Lab Chip. 11, 2282-2287 (2011).
48. Colter, D. C., Sekiya, I. & Prockop, D. J. Identification of a subpopulation of rapidly self-renewing and multipotential adult stem cells in colonies of human marrow stromal cells. Proc. Natl. Acad. Sci. U.S.A 98, 7841-7845 (2001).
49. Lee, W. C. et al. Multivariate biophysical markers predictive of mesenchymal stromal cell multipotency. Proc. Natl. Acad. Sci. U.S.A 111, E4409-E4418 (2014).
50. Yin, L. et al. Microfluidic label-free selection of mesenchymal stem cell subpopulation during culture expansion extends the chondrogenic potential: In vitro. Lab Chip 18, 878-889 (2018).
51. Wu, J., Niu, J., Li, X., Wang, X., Guo, Z., Zhang, F. TGF-β1 induces senescence of bone marrow. pdf. BMC Dev. Biol. 14, 1-9 (2014).
52. Ksiazek, K. A comprehensive review on mesenchymal stem cell growth and senescence. Rejuvenation Res. 12, 105-116 (2009).
53. Lunyak, V. V., Amaro-Ortiz, A. & Gaur, M. Mesenchymal stem cells secretory responses: Senescence messaging secretome and immunomodulation perspective. Front. Genet. 8, 1-21 (2017).
54. Hirayama, T. & Nagasawa, H. Chemical tools for detecting Fe ions. J. Clin. Biochem. Nutr. 60, 39-48 (2017).
55. Tenopoulou, M., Kurz, T., Doulias, P. T., Galaris, D. & Brunk, U. T. Does the calcein-AM method assay the total cellular ‘labile iron pool’ or only a fraction of it? Biochem. J. 403, 261-266 (2007).
56. Abbasi, U., Abbina, S., Gill, A., Bhagat, V. & Kizhakkedathu, J. N. A facile colorimetric method for the quantification of labile iron pool and total iron in cells and tissue specimens. Sci. Rep. 11, 1-12 (2021).
57. Mackenzie, E. L., Iwasaki, K. & Tsuji, Y. Intracellular iron transport and storage: From molecular mechanisms to health implications. Antioxidants Redox Signal. 10, 997-1030 (2008).
58. Denoyer, D. et al. Copper as a target for prostate cancer therapeutics: Copper-ionophore pharmacology and altering systemic copper distribution. Oncotarget 7, 37064-37080 (2016).
59. Dusek, P., Dezortova, M. & Wuerfel, J. Imaging of Iron. International Review of Neurobiology vol. 110 (2013).
60. Van Walderveen, M. A. A. et al. Hypointense lesions on T1-weighted spin-echo magnetic resonance imaging: Relation to clinical characteristics in subgroups of patients with multiple sclerosis. Arch. Neurol. 58, 76-81 (2001).
61. Golay, X. et al. Measurement of tissue oxygen extraction ratios from venous blood T2: Increased precision and validation of principle. Magn. Reson. Med. 46, 282-291 (2001).
62. Parikh, T. et al. Focal liver lesion detection and characterization with diffusion-weighted MR imaging: Comparison with standard breath-hold T2-weighted imaging. Radiology 246, 812-822 (2008).
63. Stucht, D. et al. Highest resolution in vivo human brain MRI using prospective motion correction. PLOS One 10, 1-17 (2015).
64. Carr, H. Y. & Purcell, E. M. Effects of diffusion on free precession in nuclear magnetic resonance experiments. Phys. Rev. 94, 630-638 (1954).
65. Meiboom, S. & Gill, D. Modified spin-echo method for measuring nuclear relaxation times. Rev. Sci. Instrum. 29, 688-691 (1958).
66. Bernadotte, A., Mikhelson, V. M. & Spivak, I. M. Markers of cellular senescence. Telomere shortening as a marker of cellular senescence. Aging (Albany. NY). 8, 3-11 (2016).
67. Itahana, K., Itahana, Y. & Dimri, G. P. Colorimetric detection of senescence-associated β galactosidase. Methods in Molecular Biology vol. 965 143-156 (2013).

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

1. A method of detecting senescent cells comprising:
loading a liquid sample including a plurality of cells in a sensor;
placing the sensor including the liquid sample within or nearby a detection coil of a magnetic resonance relaxometry device; and
determining a T₂ value to detect an amount of senescent cells in the liquid sample.

2. The method according to claim 1, wherein the sensor is a tube or chamber.

3. The method according to claim 1, wherein the amount of senescent cells in the liquid sample is determined relative to a reference sample.

4. The method according to claim 1, wherein the amount of senescent cells in the liquid sample is proportional to a ferritin concentration in the liquid sample.

5. The method according to claim 1, wherein determining a T₂ value to detect an amount of senescent cells includes quantifying an amount of Fe³⁺ in the liquid sample.

6. The method according to claim 1, wherein the T₁ relaxation time is used in conjunction with T₂ value to determine whether senescent cells are present in the sample.

7. The method according to claim 6, wherein determining the T₁ or T₂ value includes supplying a train of pulses over a period of less than one minute.

8. The method according to claim 6, wherein determining the T₁ or T₂ value includes obtaining and averaging 2 to 70 scans.

9. The method according to claim 1, wherein the T₂ value decreases as a number of senescent cells increases.

10. The method according to claim 1, wherein the cells include mesenchymal stromal cells.

11. The method according to claim 1, wherein the cells include hematopoietic stem cells.

12. The method according to claim 1, wherein the cells include induced pluripotent stem cells.

13. The method according to claim 1, wherein a detection region of the magnetic resonance relaxometry device comprises a volume of less than about 1 μL of the sample.

14. The method according to claim 1, wherein determining a T₂ includes measuring a content of paramagnetic or ferromagnetic ions of the cells.

15. A method according to claim 14, wherein the paramagnetic or ferromagnetic ions include copper.

16. A method of improving efficacy of stem cells or progenitor cells for patients, the method comprising:
detecting senescent cells in a sample according to the method of claim 1;
concentrating cells in the sample to reduce a number of the senescent cells in the sample.

17. The method according to claim 16, wherein concentrating cells includes passing the sample through a cell separation device, thereby separating senescent cells from non-senescent cells in the sample.

18. The method of claim 16, wherein the cell separation device includes a microfluidic spiral path.

19. A system for detecting senescent cells comprising:
a magnetic resonance relaxometry device configured to detect an amount of senescent cells in a liquid sample; and
a cell separation device for reducing the amount of senescent cells in the liquid sample based on output from the magnetic resonance relaxometry device.

20. The system of claim 19, wherein the liquid sample is contained in a microcapillary.

21. The system of claim 19, wherein the liquid sample is taken from a cell batch.

22. The system of claim 21, wherein the cell separation device removes senescent cells from the cell batch based on detecting the amount of senescent cells.

23. The system of claim 19, wherein the cell separation device includes a microfluidic cell sorter, an inertial focusing device, a microfluidic filtration device, a centrifugal flow device, a deterministic lateral displacement (DLD) chip, a tangential flow microfiltration device, or combinations thereof.

24. The system of claim 19, wherein the cell separation device includes a microfluidic spiral path device.

25. The system of claim 19, wherein the magnetic resonance relaxometry device is configured to determine a T₂ value to detect an amount of senescent cells in the liquid sample.

26. The system according to claim 19, wherein the amount of senescent cells in the liquid sample is proportional to a ferritin concentration in the liquid sample.

27. The system according to claim 19, wherein the magnetic resonance relaxometry device is configured to determine a T₂ value to detect an amount of senescent cells includes quantifying an amount of Fe³⁺ in the liquid sample.

28. The system according to claim 19, wherein the senescent cells include mesenchymal stromal cells (MSCs), hematopoietic stem cells (HSCs), or induced pluripotent stem cells (iPSCs).
